(19) United States
(12) Patent Application Publication
(10) Pub. No.: US 2013/0023459 A1
(43) Pub. Date: Jan. 24, 2013

(54) Method for Preparing a Protein Emulsion

(71) Applicant: Patrick Willibrord Koehorst, Haaksbergen (NL)

(72) Inventor: Patrick Willibrord Koehorst, Haaksbergen (NL)

(21) Appl. No.: 13/582,390

(22) PCT Filed: Mar. 1, 2011
(86) PCT No.: PCT/NL2011/050140

§ 371 Date: Oct. 10, 2012

(30) Foreign Application Priority Data
Mar. 1, 2010 (NL) 2004319

(51) Int. Cl. C12P 21/06; A23K 1/10; A23J 1/04; A23J 1/10; A61K 38/02; A23J 3/34

(52) U.S. Cl. 514/1.1; 435/68.1; 426/56; 426/7; 426/541; 426/665

(57) ABSTRACT

The present invention relates to a method for preparing a protein emulsion from bone material from which meat has been at least partially removed. The invention also relates to the protein emulsion obtained with such a method and to the use of this protein emulsion. In addition, the invention also relates to a food product comprising the protein emulsion according to the present invention.

The present invention relates to a method for preparing a protein emulsion, to the obtained protein emulsion and to the use thereof.

In slaughterhouses there remains a bone material part after slaughter and deboning of cadavers. Although most of the meat (i.e. particularly fat and protein) has been removed from the bone material, significant quantities of meat still remain attached to this bone material. This attached meat is very difficult to separate manually from the bone material.

In order to separate the meat fraction from the bone fraction it is known to make use of mechanical pressing. The bone material with the meat thereon is here first crushed, after which it is pressed under increased pressure through a sieve. Although part of the meat is in this way separated from the bone material, there is however the risk of bone splinters passing through the sieve and being included in the meat fraction. This is of course undesirable from a food safety viewpoint. In addition, a part of the meat and fat is still left on the bone after the pressing.

Another way of separating the meat fraction from the bone fraction is by means of enzymatic hydrolysis. Because the meat attached to the bone material consists substantially of protein and fat, the different fractions can be separated from each other by means of hydrolysis of the protein part. Such a method is described in the international patent publication WO2006/103628. One of the drawbacks of this method is however that only the water-soluble part of the protein hydrolysate can be used, the part of the hydrolysate which is not water-soluble, i.e. the insoluble protein fraction, cannot be used for human consumption because of its unattractive colour. Although the possible use of antioxidants is considered in WO02/065848, they are only added after the enzymatic hydrolysis. According to WO 02/065848 the reason for using antioxidants is to prevent oxidation of the fats, and not to prevent an unattractive colour of the protein fraction.

The present invention has for its object, among others, to provide a solution for the above stated problems. In a first aspect of the present invention a method is provided for preparing a protein emulsion, comprising of:
i) introducing crude bone material, water, enzymes, preferably a protease, and antioxidants into a reaction vessel; ii) enzymatic hydrolysis of the crude bone material; iii) deactivating the enzymes; and iv) substantially removing the treated bone fraction from the reaction mixture.

The term crude bone material is understood to mean bone material which still comprises residues of other biological material such as meat, blood, tendons, marrow and the like. Enzymes are understood to mean a number of enzymes, i.e. one or more enzymes, preferably proteolytic enzymes. Because use is made of antioxidants during the hydrolysis, considerably fewer taste variations and colour variations occur in the protein than in conventional methods.

The crude bone material can originate from a large number of animals. Typical examples of crude bone material can be bone material from chickens, pigs, cattle (such as cows), goats, sheep, horses. The bone material can also comprise parts of fish, molluscs and crustaceans. It is however also possible for the crude bone material to comprise a mixture of bone material of different animals. It is further recommended to first crush the bone material. A higher yield can hereby be obtained.

In order to preserve the colour of the crude bone material as well as possible the hydrolysis of the crude bone material is preferably performed under modified atmospheric conditions. An inert gas such as nitrogen or carbon dioxide, or a mixture thereof, is preferably added to the treatment space and/or the reaction vessel such that an oxygen content or O₂ is obtained which is lower than 10% oxygen, and preferably lower than 5% oxygen.

By making use of this above stated method the protein residue, of for instance the attached meat, can be removed in efficient manner from the crude bone material. By removing the (treated) bone fraction, i.e. the bone material which has been treated with the hydrolysis reaction, from the reaction mixture a so-called protein emulsion remains. In addition to water, this protein emulsion comprises water-soluble protein, water-insoluble protein, fat/oil or a mixture of these fractions. Separation of the (treated) bone material from the reaction mixture can take place using for instance a centrifuge or a decanter. Because antioxidants are used, the colour and taste of the protein fractions is of a quality such that they can be used to manufacture food products for humans and animals. It is particularly recommended to use the protein fraction as a semi-product for determined food products. It is also possible to combine the protein emulsion with a fat fraction of a different origin, for instance vegetable fats or oils.

50% or more of the proteins in the protein emulsion according to the present invention preferably has a molecular weight greater than 100000 Dalton. More than 70% of the proteins preferably has a molecular weight greater than 100000 Dalton.

It is further recommended to use one or more of the following antioxidants: sodium ascorbate, potassium nitrate, sodium nitrite, ascorbic acid, sodium isoascorbate, erythorbic acid, vitamin E, metabisulfite, rosemary extract or mixtures thereof. These antioxidants are particularly suitable for use in the present method because they are stable at increased temperature as is applied in the enzymatic hydrolysis. In addition, these antioxidants are generally recognized and are applied on large scale in normal meat processing. The quantity of antioxidants used preferably comprises about 0.1% by weight to 10% by weight of the crude bone material to be treated. The quantity of antioxidants more preferably comprises between 0.1% by weight and 1% by weight of the crude bone material to be treated.

The enzymatic hydrolysis of the crude bone material is preferably performed at a temperature between 40 and 65 degrees Celsius. The pressure can vary here between 0 and 5 bar.

Enzymes which are used are so-called proteases or proteolytic enzymes which are generally know to the skilled person. The quantity of enzymes used is preferably between 0.01% by weight and 10% by weight of the crude bone material.

When the enzymatic hydrolysis reaction has to be stopped, the temperature is preferably increased to a minimum of 80 degrees Celsius. It is also possible to inactivate the enzymes by means of a pH reduction, such as a reduction to below pH 4. It is also possible to inactivate the enzymes by increasing the pH, such as an increase to above pH 8. Another way of inactivating the enzymes is by subjecting the reaction mixture or the protein emulsion to a considerable pressure increase. The above stated techniques can also be combined with each other.

The protein emulsion obtained with the present invention can be used as food product. It is however also readily possible to use it as semi-product in the production of foods. In order to stabilize and/or make the protein emulsion cuttable use can be made of thickening agents such as starches (native or modified, or combinations thereof), carrageenans (kappa, lambda or iota, or combinations thereof), guar gum, carob bean gum, pectin, alginate, gelatin, xanthan, koniaku or combinations of these thickening agents. For stabilizing purposes use can also be made of stabilizing agents such as animal proteins (originating from meat, fish, dairy products and eggs) and vegetable proteins (originating from grain products, beans and legumes), or combinations thereof. Finally, enzymes (such as transglutaminase) can also be applied to stabilize the food product.

A subsequent aspect of the present invention relates to a protein emulsion such as can be obtained using the above stated method. The protein emulsion can here comprise fully hydrolyzed proteins, partially hydrolyzed proteins, fats, oils or combinations thereof. 50% or more of the proteins in the protein emulsion preferably have a molecular weight greater than 100000 Dalton. It is particularly recommended that more than 70% of the proteins in the protein emulsion have a molecular weight greater than 100000 Dalton.

A further aspect of the present invention relates to the use of the protein emulsion in food products for human consumption or animal consumption.

A final aspect of the present invention relates to the use of the protein emulsion in human or animal medicines.

Stated and other features of the protein product according to the present invention will be elucidated hereinbelow on the basis of an exemplary embodiment which is given only by way of example, without the invention being deemed limited thereby.

### EXAMPLE

### Production of a Chicken Product.

Chicken bones remaining after the manual deboning of chicken are used as starting material. In order to minimize the adverse effect of chicken bone marrow on the taste of the end product the bones are not reduced in size. Calculated according to the quantity of starting material, 1% nitrite brining salt and 0.1% sodium ascorbate is dissolved in the water fraction, after which the whole is mixed in a heatable vessel with suitable stirring means. Water is added in a 1:1 ratio. After 5 minutes of mixing at low temperature (T.<10° Celsius) the content of the vessel is heated to 55° Celsius. At this temperature, 0.1% (calculated according to the bone fraction) of a commercial enzyme such as Protamex® is added; mixing takes place at this temperature for 30 minutes. After this hydrolysis phase the temperature of the whole is increased to 80° Celsius for 30 minutes. Immediately after inactivation, the content of the hydrolysis vessel, while being stirred, is gradually guided over a vibration screen (Allgaier), wherein the bone material is separated from the rest of the reaction mixture, also referred to as the protein emulsion.

The present invention is not limited to the above described embodiments, the rights sought being defined rather by the following claims, within the scope of which many possible modifications can be envisaged.

1. Method for preparing a protein emulsion, comprising of:
i) introducing crude bone material, water, enzymes, preferably a protease, and antioxidants into a reaction vessel;
ii) enzymatic hydrolysis of the crude bone material;
iii) deactivating the enzymes; and
iv) substantially removing the treated bone fraction from the reaction mixture.

2. Method as claimed in claim 1, wherein the protein emulsion comprises water-soluble protein, water-insoluble protein, fat, oil or a mixture of said fractions.

3. Method as claimed in claim 1, wherein 50% or more of the proteins in the protein emulsion has a molecular weight greater than 100000 Dalton.

4. Method as claimed in claim 1, wherein the antioxidants are chosen from the group comprising sodium ascorbate, potassium nitrate, sodium nitrite, ascorbic acid, sodium isoascorbate, erythorbic acid, vitamin E, metabisulfite, rosemary extract or mixtures thereof.

5. Method as claimed in claim 1, wherein the enzymatic hydrolysis of the reaction mixture takes place at a temperature between 40 and 65 degrees Celsius and a pressure between 0 and 5 bar.

6. Method as claimed in claim 1, wherein the deactivation of the enzymes takes place by increasing the temperature of the reaction mixture above 80 degrees Celsius, by setting the pH below 4 or above 8, by applying high pressure, or by a combination of the foregoing.

7. Method as claimed in claim 1, wherein the crude bone material comprises bone material from chickens, pigs, cattle, cows, sheep, goats, horses and/or fish, molluscs and crustaceans, preferably chickens, pigs, cattle, cows, sheep, goats and/or horses.

8. Method as claimed in claim 1, wherein the quantity of antioxidants used is 0.1% by weight to 10% by weight of the crude bone material.

9. Method as claimed in claim 1, wherein the quantity of enzymes, preferably proteolytic enzymes, used is between 0.01% by weight and 10% by weight of the crude bone material.

10. Method as claimed in claim 1, wherein the protein emulsion separated from the reaction mixture is a semi-product.

11. Method as claimed in claim 10, wherein the semi-product is further processed to a food product.

12. Food product obtainable with the method as claimed in claim 11.

13. Protein emulsion obtainable using the method as claimed in claim 1.

14. Protein emulsion as claimed in claim 13, comprising fully hydrolyzed proteins, partially hydrolyzed proteins, fats, oils or combinations thereof.

15. Protein emulsion as claimed in claim 13, wherein 50% or more of the proteins in the protein emulsion has a molecular weight greater than 100000 Dalton.

16. Use of a protein emulsion as claimed in claim 13 in food products for human consumption.

17. Use of a protein emulsion as claimed in claim 13 in human or animal medicines or animal feeds.
